(19) United States
(12) Patent Application Publication
(10) Pub. No.: US 2016/0003817 A1
(43) Pub. Date: Jan. 7, 2016

(54) Rapid and sensitive analyte measurement assay

(71) Applicant: THE TRUSTEES OF PRINCETON UNIVERSITY, Princeton, NJ (US)

(72) Inventors: Stephen Y. Chou, Princeton, NJ (US); Liang-Cheng Zhou, Princeton, NJ (US)

(21) Appl. No.: 14/852,417

(22) Filed: Sep. 11, 2015

Related U.S. Application Data
(63) Continuation of application No. PCT/US2014/029675, filed on Mar. 14, 2014.
Application No. PCT/US2014/029675 is a continuation-in-part of application No. 13/838,600, filed on Mar. 15, 2013.
Application No. 13/838,600 is a continuation-in-part of application No. 13/699,270, filed on Jun. 13, 2013, now Pat. No. 9,182,338.
Application No. 13/699,270 is a continuation-in-part of application No. 13/699,270, filed on Jun. 13, 2013, now Pat. No. 9,182,338.
(60) Provisional application No. 61/800,915, filed on Mar. 15, 2013.
(60) Provisional application No. 61/622,226, filed on Apr. 10, 2012.
(60) Provisional application No. 61/347,178, filed on May 21, 2010.
(60) Provisional application No. 61/347,178, filed on May 21, 2010.

(51) Int. Cl. G01N 33/543; G01N 21/63; G01N 21/66

(52) U.S. Cl. CPC G01N 33/54393; G01N 33/5438; G01N 21/66; G01N 21/63; G01N 2201/06113

(57) ABSTRACT

This disclosure provides, among other things, a method to speed up the time in an assay, comprising: obtaining a plate comprising a local electric-field and electric-field gradient enhancement layer on a substrate surface; attaching capture agents to the surface of the enhancement layer; applying a voltage between the enhancement layer and at least one counter electrode to produce a local electric field and an electric-field gradient in the solution; and detecting binding of the target analyte to the capture agents on the plate; wherein the speed of movement of the analyte, the orientation of the analyte, the orientation of the capture agent, the speed of binding and/or the strength of binding of the analyte to the capture agent are improved by the electric field gradient and/or the electric field, and the time in detecting the analyte is reduced. Systems for performing the method are also disclosed.

### CROSS-REFERENCING

This application is a continuation-in-part of U.S. application Ser. No. 13/838,600, filed Mar. 15, 2013 (NSNR-003), which application claims the benefit of U.S. provisional application Ser. No. 61/622,226 filed on Apr. 10, 2012, and is a continuation-in-part of U.S. patent application Ser. No. 13/699,270, filed on Jun. 13, 2013, which application is a §371 filing of US2011/037455, filed on May 20, 2011, and claims the benefit of U.S. provisional application Ser. No. 61/347,178, filed on May 21, 2010;

This application is also a continuation-in-part of U.S. application Ser. No. 13/699,270, filed Jun. 13, 2013 (NSNR-001), which application is a §371 filing of international application Ser. No. US2011/037455, filed on May 20, 2011, which application claims the benefit of U.S. Provisional Patent Application Ser. No. 61/347,178 filed on May 21, 2010; and

This application is also claims the benefit of: provisional application Ser. No. 61/801,424, filed Mar. 15, 2013 (NSNR-004PRV), provisional application Ser. No. 61/801,096, filed Mar. 15, 2013 (NSNR-005PRV), provisional application Ser. No. 61/800,915, filed Mar. 15, 2013 (NSNR-006PRV), provisional application Ser. No. 61/793,092, filed Mar. 15, 2013 (NSNR-008PRV), provisional Application Ser. No. 61/801,933, filed Mar. 15, 2013 (NSNR-009PRV), provisional Application Ser. No. 61/794,317, filed Mar. 15, 2013 (NSNR-010PRV), provisional application Ser. No. 61/802,020, filed Mar. 15, 2013 (NSNR-011PRV) and provisional application Ser. No. 61/802,223, filed Mar. 15, 2013 (NSNR-012PRV), all of which applications are incorporated by reference herein for all purposes.

### STATEMENT REGARDING FEDERALLY SPONSORED RESEARCH

This invention was made with government support under Grant No. FA9550-08-1-0222 awarded by the Defense Advanced Research Project Agency (DARPA). The government has certain rights in the invention.

### BACKGROUND

The invention is related to the methods, devices, and systems that reduce the assay incubation time and improve the movements, bonding, and orientation of analytes and associated capture agents and labels, and hence assay sensing quality.

In a solid-phase assay, the capture agents immobilized on a solid surface catch and bond the analytes in solution. In conventional assay methods, the movement of an analyte in a solution is by the diffusion process, and hence the time for the immobilized capture agent to catch an analyte in a solution depends on the analyte's diffusion time. The diffusion time depends on the square of the distance that an analyte travels. Furthermore, the analyte bonding to the capture agents also needs time. The incubation time, the time needed for the capture agents to capture sufficient analytes in a solution for a sensing, depends on the analyte diffusion time and the analyte bonding time, which can be very long, several hours or longer.

One way to reduce the diffusion time is to applying an additional force to push the analytes toward to the capture agent. One way to help the bonding between the analytes and the capture agents is to align the appropriate bonding sites of these molecules in a proper orientation. Both them can be achieved by an electrical force. The larger the force, the faster the analytes move, and the more alignments of the molecules.

An electric force on a molecule can be generated in two ways: (a) using an electric field and (b) using an electric field gradient. An electric field creates a force on a molecule that has a net electrical charge. The force is proportional to the molecular net charge and the electric field. An electric field gradient creates a force on a molecule that has a net dipole moment. The force is proportional to the molecular electric dipole moment and the electric gradient.

If the molecules or particles are transported and separated due to the force caused by their charge in a uniform (i.e. homogeneous) electric field, it is termed “electrophoresis” (EP). If the molecules or particles are transported and separated due to the force caused by their electric dipole moment and the electric field gradients (i.e. in an in homogeneous electric field), it is termed “dielectrophoresis” (DEP).

Many analytes have many charges distributed around themselves, but near zero or very weak net charge. Hence, an uniform electric field will not generate sufficient force to push analytes. However, the molecules with near zero or very weak net charge often have large electrical dipole (either intrinsic or induced), hence a strong force can be generated by an electric field gradient.

A electric force with a proper direction can help the bonding between the analytes and the capture agents, and also may help the sensing signal. For example, the bonding becomes easier, if the antibody capture agents stand up with the bonding sites for catching the analytes pointing out. Similarly, a protein analyte can bond a capture agent easier, if the analytes' bonding site is oriented as the “head” of the analytes, as they are moving toward a capture agent.

The isoelectric point (pI) of a molecule and the pH level of the buffer solution pH value are two factors that together determine the net charge on a molecule. The isoelectric point (pI), sometimes abbreviated to IEP, is the pH at which a particular molecule or surface carries no net electrical charge. In a solution with a pH below their pI, the molecules carry a net positive charge; but in a solution with a pH above their pI, the molecules carry a net negative charge. Different types of the molecules can have different pI. Therefore, by controlling the pH value of the solution in the bonding process (i.e. incubation process), the quality of the bonding can be improved and the total time of the bonding process can be shortened.

There is a great need for new methods to (a) improve the molecular bonding in an assay, (b) speed the molecular movements and reduce the incubation time, (c) generate a higher electric field gradient, (d) generate a higher electric field gradient at or near the molecular bonding sites, and (e) manipulate the pH value of the solution according to the isoelectric point of molecules.

### PRIOR ARTS

Some of the prior arts use a pair of planar electrodes, without (a) using nanostructures on the electrodes and/or (b) controlling the pH value of the solution according to the isoelectric point (pI) of a molecule.

Some of the prior arts do use structured dielectric materials and/or structured electrode to generate an electric field gradient, but these structures have feature size in 10's to 100's micron size, hence leading to a small electric field gradient. Furthermore, the electric field gradient generated are at locations far away from the analytes bonding sites, and the direction of the electric field gradient often are different from direction different from the preferred direction for the analyte movement and/or bonding.

When a large DEP force is needed, prior arts have to apply very high electric field and gradient, that can cause molecular breakdown.

### SUMMARY

The following brief summary is not intended to include all features and aspects of the present invention, nor does it imply that the invention must include all features and aspects discussed in this summary.

The invention is related to the methods, devices, and systems that reduce the assay incubation time and improve the movements, bonding, and orientation of analytes and associated capture agents and labels, and hence assay sensing quality. The analyte include proteins, peptides, DNA, RNA, nucleic acid, small molecules, cells, nanoparticles with different shapes.

One embodiment of the invention is to use the nanostructured electric field and gradient enhancement layer 201 on the assay plate 202.

Another embodiment of the invention is that the EFGE layer 201 has nanoscale metal-dielectric/semiconductor-metal structures 100, 400, which enhances the local surface electric field and gradient. The regions on the EFGE layer 201 with the most enhanced local electric field and gradient where are the sharp (i.e. large curvature) edges of a metal structure and the between a small gaps of the two metal structures 130 and 150, 430 and 450. The invention includes several different EFGE layer structures.

Another embodiment of the invention is to immobilize the capture agents 160 on the surface of the electric field and gradient enhancement (EFGE) layer 201.

Another embodiment of the invention is to immobilize, in many cases, the capture agents 160 only in the regions on the surface of the EFGE layer 201 where the electric field gradient and/or the electric field are the highest.

Another embodiment of the invention is that the EFGE layer surface 201 itself serves as one of the electrode for electric biasing, hence less interfered by the solution.

Another embodiment of the invention is to use light, rather the electric bias, to create the electric field and gradient on the EFGE layer surface 201.

Another embodiment of the invention is that both electrical bias and the light are used together or alternatively to create electric field and gradient on the surface of the EFGE surface 201.

Another embodiment of the invention is that the same EFGE layer 201 also can directly amplify the sensing signal as well.

Another embodiment of the invention is that the control of the pH value of the solution according to isoelectric points of the analytes are used together the electrical bias and/or the light.

Another embodiment of the invention is that the EFEG layer 201 can used for microliter multi-well plates or microfluidic channels 207.

### BRIEF DESCRIPTION OF THE DRAWINGS

The skilled artisan will understand that the drawings, described below, are for illustration purposes only. The drawings are not intended to limit the scope of the present teachings in any way. Some of the drawings are not in scale.

FIG. 1. Schematics of the device and systems for the method of enhancing assay sensing and reducing assay incubation time by electric bias, local electric field and gradient enhancing nanostructures, and ph value control. The sensing amplification layer (SAL) 201 is served as one electrode while another conducting board 203 is served as the counter electrode for applying electric field. The voltage bias between the electrodes can be DC or AC and is supplied by the power supply, 204.

FIG. 2. One embodiment of the EFGE enhancement layer, Disk-on-Pillar (DoP) structure 400, (a) overview of general structure. (b) Cross-section of one embodiment where the back metallic film is around and next to the pillars which are dielectric or semiconductor. (c, d, e) cross-section of another embodiment, where the metallic film is a sheet of film go under the disk, but the pillars have different lateral dimension than that of the disks.

FIG. 3. Another embodiment of the EFGE enhancement layer, Disk-coupled dots-on-pillar antenna array (D2PA) plate 100. (a) Schematic (overview) of D2PA plate without an immunoassay. D2PA has an array of dense three-dimensional (3D) resonant cavity nanoantennas (formed by the gold disks 130 on top of periodic nonmetallic pillars 120 and the gold backplane 150 on the pillar foot) with dense plasmonic nanodots 140 inside, and couples the metallic components through nanogaps. (b) Schematic of the D2PA (cross-section), consisting of a self-assembled monolayer (SAM) of adhesion layer. (c) Illustration of the process of coating SAM on D2PA (cross-section). The SAM is selectively coated on D2PA components through specific chemical binding effect.

FIG. 4. Schematics of the electric fields and electric filed gradients in the regions near a D2PA plate 100. (A) Schematic (overview) of D2PA plate without an immunoassay. (B) Electric field line near the nanostructures of D2PA when an external electric field is applied. High electric field gradients at the edges of the metallic materials and between the gaps. (C) The corresponding of Dielectrophoresis (DEP) force direction felt by a biomolecules near the D2PA, due to the high gradient of electric field. (D) Electric field line near the nanostructures of D2PA nanostructures that are without nanodots between the cavity formed by top disk and backplane. (E) Electric field line near the nanostructures of Disk-on-pillar (DoP) when an external electric field is applied.

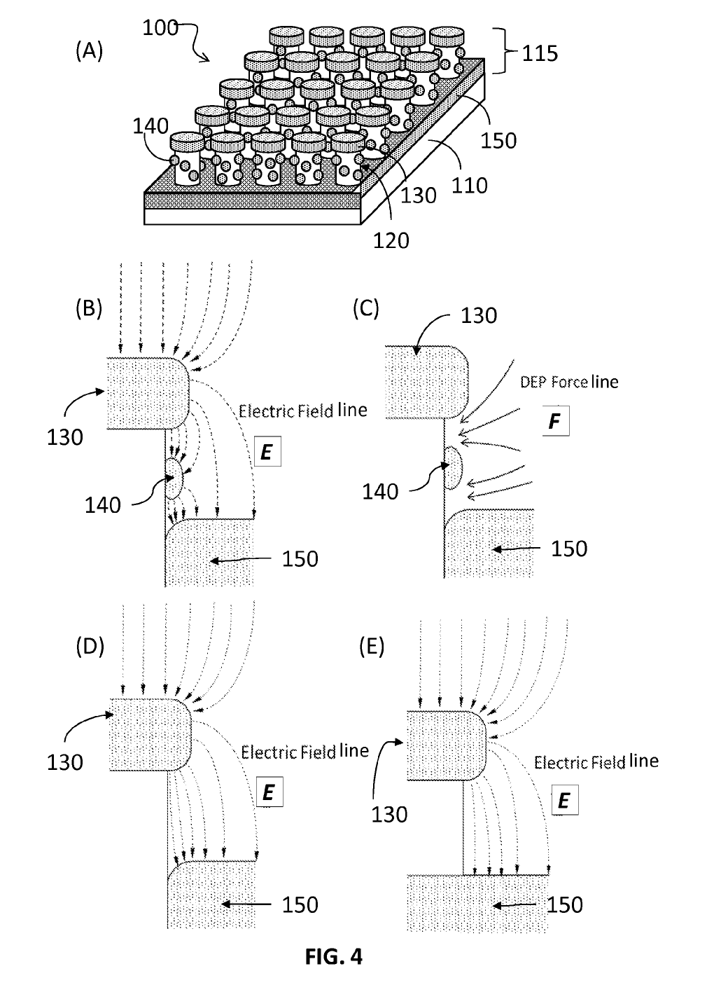

FIG. 5. Schematics of random metallic islands separated with a metal planar electrode by a thin dielectrics.

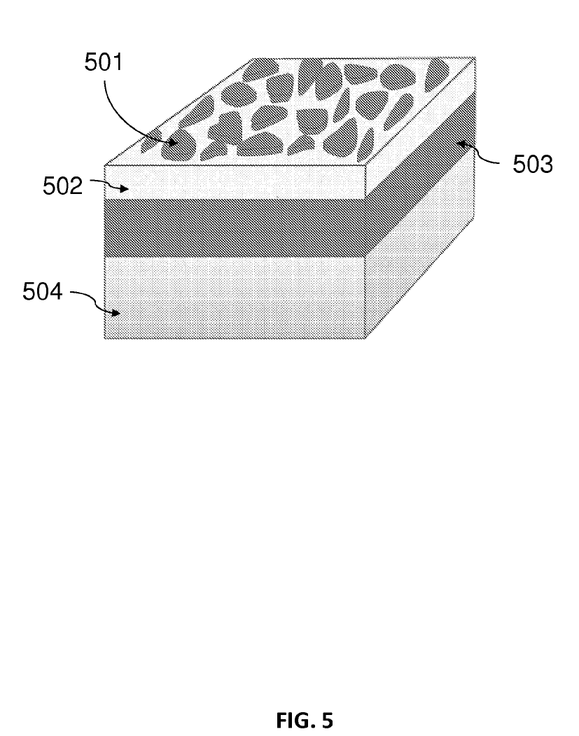

FIG. 6. Schematics of different ways to position the electrodes. (A) The counter electrodes that can be placed vertically towards the EFGE layer, as well as in the horizontal direction across the substrate. And (b) the counter electrode is in-plane with the EFGE layer.

FIG. 7. Illustration of an embodiment of an E-field assisted immunoassay platform. The voltage supplied between the “sensing amplification layer” (SAL) and a counter electrode the top conducting board and Plasmonic Nanostructures is between 0.1 V to 1000V. The SAL enhances local E-field and E-field gradient, which in turn can be applied during the reading to further enhance the assaying properties, including improve the sensing sensitivity and reducing incubation time.

FIG. 8 schematically illustrates an exemplary antibody detection assay.

FIG. 9 schematically illustrates an exemplary nucleic acid detection assay.

FIG. 10. Graph showing fluorescence intensity of 10 pM immunoassay incubated in DC field within 160s. The dashed line is the fluorescence signal intensity of the identical immunoassay performed without DC-field and using 1 hour incubation time.

FIG. 11. Under a certain voltage, the electric field and electric field gradient can help the attachment and alignment of analytes, which in turn can improve sensing sensitivity. (a) A negatively charged IgG. Though the overall charge is negative, the charge distribution on the antibodies is not uniform. (b) Schematics of the electrochemical deposition of oriented antibodies. The Au electrode is functioned with DSU monolayer.

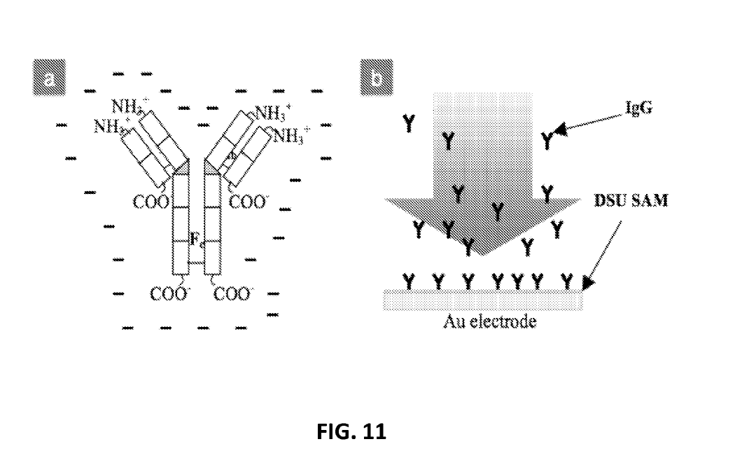

Corresponding reference numerals indicate corresponding parts throughout the several figures of the drawings. It is to be understood that the drawings are for illustrating the concepts set forth in the present disclosure and are not to scale.

Before any embodiments of the invention are explained in detail, it is to be understood that the invention is not limited in its application to the details of construction and the arrangement of components set forth in the following description or illustrated in the drawings.

### DEFINITIONS

Before describing exemplary embodiments in greater detail, the following definitions are set forth to illustrate and define the meaning and scope of the terms used in the description.

The term “electrical bias” refers to an electric voltage is applied between two points.

The term “molecular adhesion layer” refers to a layer or multilayer of molecules of defined thickness that comprises an inner surface that is attached to the nanodevice and an outer (exterior) surface can be bound to capture agents.

The term “capture agent-reactive group” refers to a moiety of chemical function in a molecule that is reactive with capture agents, i.e., can react with a moiety (e.g., a hydroxyl, sulfhydryl, carboxy or amine group) in a capture agent to produce a stable strong, e.g., covalent bond.

The term “capture agent” as used herein refers to an agent that binds to a target analyte through an interaction that is sufficient to permit the agent to bind and concentrate the target molecule from a heterogeneous mixture of different molecules. The binding interaction is typically mediated by an affinity region of the capture agent. Typical capture agents include any moiety that can specifically bind to a target analyte. Certain capture agents specifically bind a target molecule with a dissociation constant (K_{D}) of less than about 10⁻⁶ M (e.g., less than about 10⁻⁷M, less than about 10⁻⁸M, less than about 10⁻⁹M, less than about 10⁻¹⁰ M, less than about 10⁻¹¹ M, less than about 10⁻¹² M, to as low as 10⁻¹⁶ M) without significantly binding to other molecules. Exemplary capture agents include proteins (e.g., antibodies), and nucleic acids (e.g., oligonucleotides, DNA, RNA including aptamers).

The terms “specific binding” and “selective binding” refer to the ability of a capture agent to preferentially bind to a particular target molecule that is present in a heterogeneous mixture of different target molecule. A specific or selective binding interaction will discriminate between desirable (e.g., active) and undesirable (e.g., inactive) target molecules in a sample, typically more than about 10 to 100-fold or more (e.g., more than about 1000- or 10,000-fold).

The term “protein” refers to a polymeric form of amino acids of any length, i.e. greater than 2 amino acids, greater than about 5 amino acids, greater than about 10 amino acids, greater than about 20 amino acids, greater than about 50 amino acids, greater than about 100 amino acids, greater than about 200 amino acids, greater than about 500 amino acids, greater than about 1000 amino acids, greater than about 2000 amino acids, usually not greater than about 10,000 amino acids, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; fusion proteins with detectable fusion partners, e.g., fusion proteins including as a fusion partner a fluorescent protein, β-galactosidase, luciferase, etc.; and the like. Also included by these terms are polypeptides that are post-translationally modified in a cell, e.g., glycosylated, cleaved, secreted, prenylated, carboxylated, phosphorylated, etc., and polypeptides with secondary or tertiary structure, and polypeptides that are strongly bound, e.g., covalently or non-covalently, to other moieties, e.g., other polypeptides, atoms, cofactors, etc.

The term “antibody” is intended to refer to an immunoglobulin or any fragment thereof, including single chain antibodies that are capable of antigen binding and phage display antibodies).

The term “nucleic acid” and “polynucleotide” are used interchangeably herein to describe a polymer of any length composed of nucleotides, e.g., deoxyribonucleotides or ribonucleotides, or compounds produced synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions.

The term “complementary” as used herein refers to a nucleotide sequence that base-pairs by hydrogen bonds to a target nucleic acid of interest. In the canonical Watson-Crick base pairing, adenine (A) forms a base pair with thymine (T), as does guanine (G) with cytosine (C) in DNA. In RNA, thymine is replaced by uracil (U). As such, A is complementary to T and G is complementary to C. Typically, “complementary” refers to a nucleotide sequence that is fully complementary to a target of interest such that every nucleotide in the sequence is complementary to every nucleotide in the target nucleic acid in the corresponding positions. When a nucleotide sequence is not fully complementary (100% complementary) to a non-target sequence but still may base pair to the non-target sequence due to complementarity of certain stretches of nucleotide sequence to the non-target sequence, percent complementarily may be calculated to assess the possibility of a non-specific (off-target) binding. In general, a complementary of 50% or less does not lead to non-specific binding. In addition, a complementary of 70% or less may not lead to non-specific binding under stringent hybridization conditions.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” as used herein denotes single stranded nucleotide multimers of from about 10 to 200 nucleotides and up to 300 nucleotides in length, or longer, e.g., up to 500 nt in length or longer. Oligonucleotides may be synthetic and, in certain embodiments, are less than 300 nucleotides in length.

The term “attaching” as used herein refers to the strong, e.g, covalent or non-covalent, bond joining of one molecule to another.

The term “surface attached” as used herein refers to a molecule that is strongly attached to a surface.

The term “sample” as used herein relates to a material or mixture of materials containing one or more analytes of interest. In particular embodiments, the sample may be obtained from a biological sample such as cells, tissues, bodily fluids, and stool. Bodily fluids of interest include but are not limited to, amniotic fluid, aqueous humour, vitreous humour, blood (e.g., whole blood, fractionated blood, plasma, serum, etc.), breast milk, cerebrospinal fluid (CSF), cerumen (earwax), chyle, chime, endolymph, perilymph, feces, gastric acid, gastric juice, lymph, mucus (including nasal drainage and phlegm), pericardial fluid, peritoneal fluid, pleural fluid, pus, rheum, saliva, sebum (skin oil), semen, sputum, sweat, synovial fluid, tears, vomit, urine and exhaled condensate. In particular embodiments, a sample may be obtained from a subject, e.g., a human, and it may be processed prior to use in the subject assay. For example, prior to analysis, the protein/nucleic acid may be extracted from a tissue sample prior to use, methods for which are known. In particular embodiments, the sample may be a clinical sample, e.g., a sample collected from a patient.

The term “analyte” refers to a molecule (e.g., a protein, nucleic acid, or other molecule) that can be bound by a capture agent and detected.

The term “assaying” refers to testing a sample to detect the presence and/or abundance of an analyte.

As used herein, the terms “determining,” “measuring,” and “assessing,” and “assaying” are used interchangeably and include both quantitative and qualitative determinations.

As used herein, the term “light-emitting label” refers to a label that can emit light when under an external excitation. This can be luminescence. Fluorescent labels (which include dye molecules or quantum dots), and luminescent labels (e.g., electro- or chemi-luminescent labels) are types of light-emitting label. The external excitation is light (photons) for fluorescence, electrical current for electroluminescence and chemical reaction for chemi-luminscence. An external excitation can be a combination of the above.

The phrase “labeled analyte” refers to an analyte that is detectably labeled with a light emitting label such that the analyte can be detected by assessing the presence of the label. A labeled analyte may be labeled directly (i.e., the analyte itself may be directly conjugated to a label, e.g., via a strong bond, e.g., a covalent or non-covalent bond), or a labeled analyte may be labeled indirectly (i.e., the analyte is bound by a secondary capture agent that is directly labeled).

The term “hybridization” refers to the specific binding of a nucleic acid to a complementary nucleic acid via Watson-Crick base pairing. Accordingly, the term “in situ hybridization” refers to specific binding of a nucleic acid to a metaphase or interphase chromosome.

The terms “hybridizing” and “binding”, with respect to nucleic acids, are used interchangeably.

The term “capture agent/analyte complex” is a complex that results from the specific binding of a capture agent with an analyte. A capture agent and an analyte for the capture agent will usually specifically bind to each other under “specific binding conditions” or “conditions suitable for specific binding”, where such conditions are those conditions (in terms of salt concentration, pH, detergent, protein concentration, temperature, etc.) which allow for binding to occur between capture agents and analytes to bind in solution. Such conditions, particularly with respect to antibodies and their antigens and nucleic acid hybridization are well known in the art (see, e.g., Harlow and Lane (Antibodies: A Laboratory Manual Cold Spring Harbor Laboratory, Cold Spring Harbor, N.Y. (1989) and Ausubel, et al, Short Protocols in Molecular Biology, 5th ed., Wiley & Sons, 2002).

The term “specific binding conditions” as used herein refers to conditions that produce nucleic acid duplexes or protein/protein (e.g., antibody/antigen) complexes that contain pairs of molecules that specifically bind to one another, while, at the same time, disfavor to the formation of complexes between molecules that do not specifically bind to one another. Specific binding conditions are the summation or combination (totality) of both hybridization and wash conditions, and may include a wash and blocking steps, if necessary.

For nucleic acid hybridization, specific binding conditions can be achieved by incubation at 42° C. in a solution: 50% formamide, 5×SSC (150 mM NaCl, 15 mM trisodium citrate), 50 mM sodium phosphate (pH7.6), 5×Denhardt's solution, 10% dextran sulfate, and 20 μg/ml denatured, sheared salmon sperm DNA, followed by washing the filters in 0.1×SSC at about 65° C.

For binding of an antibody to an antigen, specific binding conditions can be achieved by blocking a substrate containing antibodies in blocking solution (e.g., PBS with 3% BSA or non-fat milk), followed by incubation with a sample containing analytes in diluted blocking buffer. After this incubation, the substrate is washed in washing solution (e.g. PBS+TWEEN 20) and incubated with a secondary capture antibody (detection antibody, which recognizes a second site in the antigen). The secondary capture antibody may conjugated with an optical detectable label, e.g., a fluorophore such as IRDye800CW, Alexa 790, Dylight 800. After another wash, the presence of the bound secondary capture antibody may be detected. One of skill in the art would be knowledgeable as to the parameters that can be modified to increase the signal detected and to reduce the background noise.

The term “a secondary capture agent” which can also be referred to as a “detection agent” refers a group of biomolecules or chemical compounds that have highly specific affinity to the antigen. The secondary capture agent can be strongly linked to an optical detectable label, e.g., enzyme, fluorescence label, or can itself be detected by another detection agent that is linked to an optical detectable label through bioconjugatio (Hermanson, “Bioconjugate Techniques” Academic Press, 2nd Ed., 2008).

The term “biotin moiety” refers to an affinity agent that includes biotin or a biotin analogue such as desthiobiotin, oxybiotin, 2′-iminobiotin, diaminobiotin, biotin sulfoxide, biocytin, etc. Biotin moieties bind to streptavidin with an affinity of at least 10-8M. A biotin affinity agent may also include a linker, e.g., -LC-biotin, -LC-LC-Biotin, -SLC-Biotin or -PEGn-Biotin where n is 3-12.

The term “streptavidin” refers to both streptavidin and avidin, as well as any variants thereof that bind to biotin with high affinity.

The term “marker” refers to an analyte whose presence or abundance in a biological sample is correlated with a disease or condition.

The term “bond” includes covalent and non-covalent bonds, including hydrogen bonds, ionic bonds and bonds produced by van der Waal forces.

The term “amplify” refers to an increase in the magnitude of a signal, e.g., at least a 10-fold increase, at least a 100-fold increase at least a 1,000-fold increase, at least a 10,000-fold increase, or at least a 100,000-fold increase in a signal.

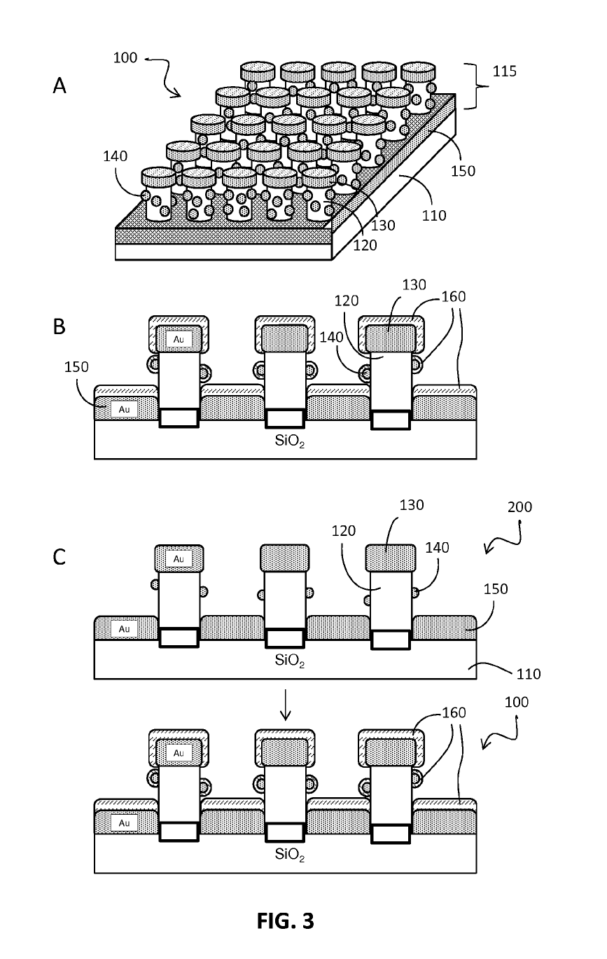

The terms “disk-coupled dots-on-pillar antenna array” and “D2PA” as used herein refer to the device illustrated in FIG. 3, where the array 100 comprises: (a) substrate 110; and (b) a D2PA structure, on the surface of the substrate, comprising one or a plurality of pillars 115 extending from a surface of the substrate, wherein at least one of the pillars comprises a pillar body 120, metallic disc 130 on top of the pillar, metallic back plane 150 at the foot of the pillar, the metallic back plane covering a substantial portion of the substrate surface near the foot of the pillar; metallic dot structure 130 disposed on sidewall of the pillar. The D2PA amplifies a light signal that is proximal to the surface of the D2PA. The D2PA enhances local electric field and local electric field gradient in regions that is proximal to the surface of the D2PA. The light signal includes light scattering, light diffraction, light absorption, nonlinear light generation and absorption, Raman scattering, chromaticity, luminescence that includes fluorescence, electroluminescence, chemiluminescence, and electrochemiluminescence.

A D2PA array may also comprise a molecular adhesion layer that covers at least a part of said metallic dot structure, said metal disc, and/or said metallic back plane and, optionally, a capture agent that specifically binds to an analyte, wherein said capture agent is linked to the molecular adhesion layer of the D2PA array. The nanosensor can amplify a light signal from an analyte, when said analyte is bound to the capture agent. One preferred SAL embodiment is that the dimension of one, several or all critical metallic and dielectric components of SAL are less than the wavelength of the light in sensing. Details of the physical structure of disk-coupled dots-on-pillar antenna arrays, methods for their fabrication, methods for linking capture agents to disk-coupled dots-on-pillar antenna arrays and methods of using disk-coupled dots-on-pillar antenna arrays to detect analytes are described in a variety of publications including WO2012024006, WO2013154770, Li et al (Optics Express 2011 19, 3925-3936), Zhang et al (Nanotechnology 2012 23: 225-301); and Zhou et al (Anal. Chem. 2012 84: 4489) which are incorporated by reference for those disclosures.

Other specific binding conditions are known in the art and may also be employed herein.

It must be noted that as used herein and in the appended claims, the singular forms “a”, “an”, and “the” include plural referents unless the context clearly dictates otherwise, e.g., when the word “single” is used. For example, reference to “an analyte” includes a single analyte and multiple analytes, reference to “a capture agent” includes a single capture agent and multiple capture agents, and reference to “a detection agent” includes a single detection agent and multiple detection agents.

### DETAILED DESCRIPTION OF EXEMPLARY EMBODIMENTS

The following detailed description illustrates some embodiments of the invention by way of example and not by way of limitation.

The invention is related to the methods, devices, and systems that reduce the assay incubation time and improve the movements, bonding, and orientation of analytes and associated capture agents and labels, and hence assay sensing quality. The analyte include proteins, peptides, DNA, RNA, nucleic acid, small molecules, cells, nanoparticles with different shapes. The sensing includes the sensing of the electromagnetic signal, including electrical and optical signals with different frequencies, light intensity, fluorescence, chromaticity, luminescence (electrical and chemo-luminescence), Raman scattering, time resolved signal (including blinking), that are related to the captured analytes.

One key embodiment of the invention is to use the nanostructured electric field and gradient enhancement layer 201 on the assay plate 202.

Another key embodiment of the invention is that the EFGE layer 201 has nanoscale metal-dielectric/semiconductor-metal structures 100, 400, which enhances the local surface electric field and gradient. The regions on the EFGE layer 201 with the most enhanced local electric field and gradient where are the sharp (i.e. large curvature) edges of a metal structure and the between a small gaps of the two metal structures 130 and 150, 430 and 450. The invention includes several different EFGE layer structures.

Another key embodiment of the invention is to immobilize the capture agents 160 on the surface of the electric field and gradient enhancement (EFGE) layer 201.

Another key embodiment of the invention is to immobilize, in many cases, the capture agents only in the regions on the surface of the EFGE layer where the electric field gradient and/or the electric field are the highest.

Another key embodiment of the invention is that the EFGE layer surface itself serves as one of the electrode for electric biasing, hence less interfered by the solution.

Another key embodiment of the invention is to use light, rather the electric bias, to create the electric field and gradient on the EFGE layer surface.

Another embodiment of the invention is that both electrical bias and the light are used together or alternatively to create electric field and gradient on the surface of the EFGE surface.

Another embodiment of the invention is that the same EFGE layer also can directly amplify the sensing signal as well.

Another embodiment of the invention is that the control of the pH value of the solution according to isoelectric points of the analytes are used together the electrical bias and/or the light.

Another embodiment of the invention is that the EFEG layer can used for microliter multi-well plates or microfluidic channels.

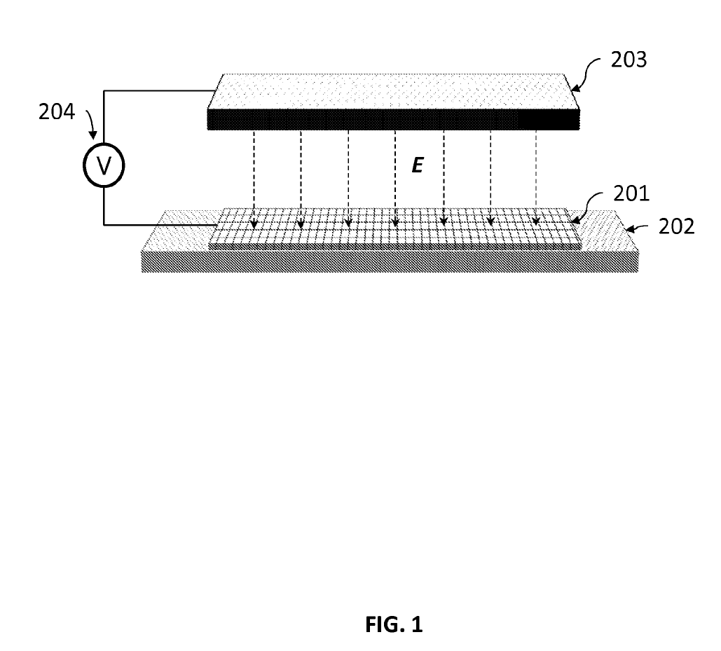

In one of embodiment of the methods to reduce assay incubation time and improve the movement, orientation, or bonding of analytes in an analyte sensing, comprises (a) obtaining a plate 200 comprising a local electric-field and electric-field gradient enhancement layer 201 on a substrate surface 202; (b) attaching capture agents on surface of the enhancement layer 201; (c) applying a voltage between the enhancement layer and at least one counter electrode 203 to produce a local electric field and electric-field gradient (FIG. 1); and (d) binding the analytes in a solution to the capture agents 160 (FIG. 3); wherein the movement speed of the analyte, the orientation of the analyte, the orientation of the capture agent, or the binding speed and/or strength of the analyte to the capture agent are improved by the electric field gradient and/or the electric field. The same EFGE layer 201 also can directly amplify the sensing signal as well.

The analyte include proteins, peptides, DNA, RNA, nucleic acid, small molecules, cells, nanoparticles with different shapes. The sensing includes the sensing of the electromagnetic signal, including electrical and optical signals with different frequencies light scattering, light diffraction, light absorption, nonlinear light generation and absorption, light intensity, fluorescence, chromaticity, luminescence (electrical and chemo-luminescence), Raman scattering, time resolved signal (including blinking), that are related to the captured analytes.

### The Local Electric Field and Field Gradient Enhancement (EFGE) Layer

The invention includes several embodiments of the local electric field and electric-field gradient enhancement (EFGE) layer 201, that have nanoscale metal-dielectric/semiconductor-metal structures, which enhances the local surface electric field and gradient. The regions on the EFGE layer 201 with the most enhanced local electric field and gradient where are the sharp (i.e. large curvature) edges of a metal structure and the between a small gaps of the two metal structures. The highest enhancement regions are those having both the sharp edges and the small gaps. The small gaps mean the distance between two metallic structures of 0.5 to 100 nm, preferably 0.5 nm to 25 nm. A preferred EFGE layer 201 should have as many the metallic sharp edges and the small gaps as possible. This requires having dense of metallic nanostructures with small gaps apart. The invention includes several different EFGE layer structures. Furthermore, the EFGE layer itself can be further improved by a process that can further cover the portions of the metallic materials that do not have sharp edges and small gaps, as described in U.S. provisional application Ser. No. 61/801,424, filed on Mar. 15, 2013, and copending PCT application entitled “Methods for enhancing assay sensing properties by selectively masking local surfaces”, filed on Mar. 15, 2014, which are incorporated by reference

One embodiment of the EFGE layer comprises a or a plural of metallic discs and a significantly continuous metallic film, wherein a substantial portion of the metallic disc has a separation from the metallic film. For enhancing the electric field and electric field gradient generated by an electric bias (DC or AC), the separation (i.e. the distance between two metallic structures) should be 0.5 to 100 nm, preferably 0.5 nm to 25 nm, and the dimensions of the disks be submicron, preferably, less than 500 nm or 100 nm smaller. For enhancing sensing signals related to light, the separation and the dimensions of the disks are less than the wavelength of the light used in sensing. Thus such structures have many sharp edges in the metallic materials and many small gaps (i.e. distance) between metallic structures.

### Examples for EFGE Structures-1: Disk on Pillar (DoP)

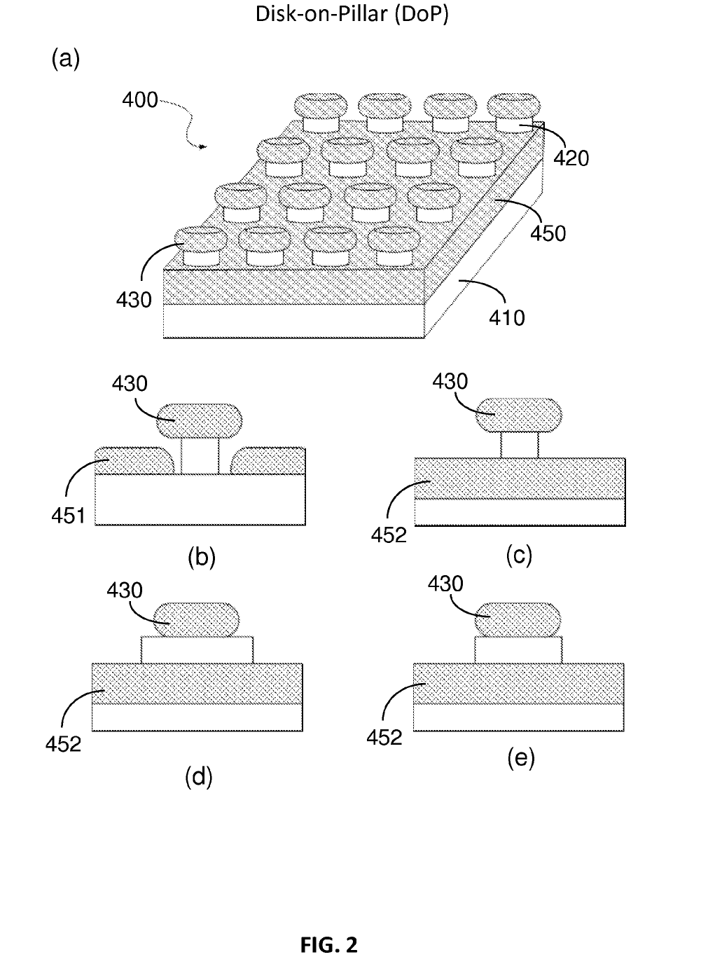

Several examples of the embodiments, disk on pillar (DoP) 400, shown in FIG. 2, comprise a substrate 410; substantially continuous metallic film 420, one or a plurality of pillars extending from a surface of the substrate, wherein at least one of the pillars comprises a pillar body 420, metallic disc 430 on top of the pillar, and metallic backplane 450. The metallic back plane can be either type A 451: at the foot of the pillar covering a substantial portion of the substrate surface near the foot of the pillar; or type B 452: a sheet of film go under the pillar. The discs can have a lateral dimension either larger (preferred) or smaller or the same as the pillars.

For enhancing light of a wavelength of 400 nm to 1,000 nm (visible to near-infra-red), the separation is 0.5 to 30 nm, the average disc's lateral dimension is from 20 nm to 250 nm, and the disk thickness is from 10 nm to 60 nm, depending upon the light wavelength used in sensing.

### Examples for EFGE Structures-2: D2PA

With reference to FIG. 13, a D2PA plate is a plate with a surface structure, termed “disk-coupled dots-on-pillar antenna array”, (D2PA), 100 comprising: (a) substrate 110; and (b) a D2PA structure, on the surface of the substrate, comprising one or a plurality of pillars 115 extending from a surface of the substrate, wherein at least one of the pillars comprises a pillar body 120, metallic disc 130 on top of the pillar, metallic backplane 150 at the foot of the pillar, the metallic back plane covering a substantial portion of the substrate surface near the foot of the pillar; metallic dot structure 140 disposed on sidewall of the pillar. The D2PA amplifies a light signal that is proximal to the surface of the D2PA. The D2PA enhances local electric field and local electric field gradient in regions that is proximal to the surface of the D2PA.

The light signal includes light scattering, light diffraction, light absorption, nonlinear light generation and absorption, Raman scattering, chromaticity, luminescence that includes fluorescence, electroluminescence, chemiluminescence, and electrochemiluminescence.

### General Shapes and Dimensions.

In some embodiments, the dimensions of one or more of the parts of the pillars or a distance between two components may be that is less than the wavelength of the amplified light. For example, the lateral dimension of the pillar body 120, the height of pillar body 120, the dimensions of metal disc 130, the distances between any gaps between metallic dot structures 140, the distances between metallic dot structure 140, and metallic disc 130 may be smaller than the wavelength of the amplified light. In some embodiments, the metallic dots are not used, just the metallic disks and the metallic backplane separated by a gap.

As illustrated in FIG. 2, the pillars may be arranged on the substrate in the form of an array. In particular cases, the nearest pillars of the array may be spaced by a distance that is less than the wavelength of the light. The pillar array can be periodic and aperiodic.

### Metallic Disc's's Dimensions for all EFGE Layers.

The disk array can be periodic 430 and aperiodic 501. The metallic disks in all embodiments have a shape selected from the group of shapes consisting of round, polygonal, pyramidal, elliptical, elongated bar shaped, or any combination thereof. Each disk may have the same, similar or different shapes with the other disks. The metallic disc on the top of each pillar can have a shape of rounded, pointed (as in the form of a pyramid or cone), polygonal, elliptical, elongated bar, polygon, other similar shapes or combinations thereof. The metallic disc lateral dimension and thickness should be less than the light amplified wavelength. Depending upon the amplified light wavelength, a lateral dimension of each disc can be chosen from 4 nm to 1500 nm, and a thickness of the disc is from 1 nm to 500 nm. The shape of each disc can be the same as, smaller, or larger, or different from, the shape of the top surface of the associated pillar on which it is disposed. The shape difference can be various from 0 to 200 nm depending the working wavelength.

Pillar's Materials and Dimensions for all EFGE Layers with Pillars.

The pillar array can be periodic and aperiodic. The pillar bodies on the top layer of the substrate may be formed from an insulating material, but may be semiconductors. Exemplary materials for the formation of the pillars are dielectrics: silicon-dioxide, silicon-nitride, hafnium oxide (HfO), Aluminum oxide (AlO) or semiconductors: silicon, GaAs, and GaN. Once formed, the pillars may have sidewalls which are columnar (straight), sloped, curved, or any combination thereof. The shape of the top surface of the pillar can be round, a point (of a pyramid), polygon, elliptical, elongated bar, polygon, other similar shapes or combinations thereof. The height of each pillar may be chosen from 5 nm to 300 nm.

The lateral dimension of each pillar should be less the amplified light wavelength, and should be chosen from 5 nm to 8,000 nm, according the amplified light wavelength. The spacing between the pillars in the array can be periodic or aperiodic. The preferred spacing should be less than amplified light wavelength. For some applications, a periodic period is preferred and the period is chosen to maximize the light absorption and radiation, which is light wavelength dependent. The spacing (pitch) between adjacent pillars in the array may be from 4 nm to 4000 nm.

### Metallic Backplane's Materials and Dimensions for all EFGE Layers:

The metallic backplane 150, 450, 503 works together with the metallic disks to form a light cavity. In the embodiment, the metallic back plane defines a metallic layer on the substrate with a hole for each pillar. The hole size should be less than the amplified light wavelength. The thickness of the metallic back plane is selected to be from 1 nm to 2000 nm, with a thickness in the range of 50 nm-200 nm preferred. The material of the metallic back plane can be selected from the same group as is used to form the metallic disc described above, but for a given D2PA structure, the metallic back plane can be formed from either the same or a different material as that used to form the discs. The D2PA nanodevice of any prior claim, wherein said pillar has a sidewall surface that is columnar, sloped, or curved.

Metallic Dots' Materials and Dimensions for all EFGE Layers with Dots.

Disposed on the sidewalls of each pillar between the metallic disc and the metallic back plane, the metallic dots 140 have shapes which are approximately spherical, discs-like, polygonal, elongated, other shapes or combinations thereof. The metallic dots 140 on a pillar may all have approximately the same shape, or may be individually varied. The dimensions of the metallic dots should be smaller than the amplified light wavelength, and are, depending the amplified light wavelength, preferably between 3 nm to 600 nm, and may be different in three dimensions. In some embodiments, the gaps between the neighboring metallic dots and the gap between the disc and adjacent metallic dots is between 0.5 nm to 200 nm. For many applications, a small gap is preferred to achieve a stronger enhancement of the signals. The gaps may be varied between each metallic dot on a pillar.

### Metallic Materials for all EFGEs:

The metallic materials for the metallic disks, backplanes, and dots are chosen from (a) single element metal, such as gold, silver, copper, aluminum, nickels; (b) a combination of the multiplayer and/or multilayer of the single metals; (c) metallic alloys; (d) semiconductors, (e) any other materials that generate plasmons at the amplified light wavelength, or (f) any combination of (a), (b), (c), (d) and (e). Each of the metallic disks, backplane, and dots use the same metallic materials as the others or different metallic materials.

### Substrates for all EFGEs.

The substrate 110, 410, 504 offer physical support to the D2PA and should be any materials, as long as it does not generate chemical and electromagnetic interference to the D2PA amplification. The substrate also can be in many different forms: thin film (membrane) and thick plate, flexible and rigid. The substrate may be made of a dielectric (e.g., SiO₂) although other materials may be used, e.g., silicon, GaAs, polydimethylsiloxane (PDMS), poly(methyl methacrylate) (PMMA).

### Preferred D2PA Embodiments.

All dimensions of the critical elements of D2PA are less the wavelength of the light. The metallic materials are selected from gold, silver, cooper, and aluminum and their alloys. In one embodiment that is configured for enhance light at a wavelength of ˜800 nm, the D2PA nanostructure may be composed of a periodic non-metallic (e.g. dielectric or semiconductor) pillar array (200 nm pitch and ˜100 nm diameter), a metallic disk on top of each pillar, a metallic backplane on the foot of the pillars, metallic nanodots randomly located on the pillar walls, and nanogaps between these metal components. The metallic disk has ˜120 nm diameter and is slightly larger than the diameter of the pillar, hence having an overhang. The disk array and the backplane (both are 40 nm thick) form a 3D cavity antenna that can efficiently traps the excitation light vertically and laterally. The height of the pillar is ˜50 nm and hence the nearest distance between the metallic disk and the metallic backplane is about 10 nm. The nearest distance, often termed “nanogap”, is preferred as small as possible for a higher enhancement. Each pillar has about 3 to 30 nanodots depending upon the pillar geometry and fabrication processing conditions; and the pillar density is 2.5×10⁹ pillars/cm². Again, In some embodiments, the metallic dots are not used, just the metallic disks and the metallic backplane separated by a gap.

### Other EFGE Layers.

Another embodiment of the sensing implication surface comprises a or a plural of metallic discs on a substrate and the average disc's lateral dimension of from 20 nm to 250 nm, and has at least a gap of 0.5 to 30 nm between the two adjacent discs.

### Capture Agents Attachments.

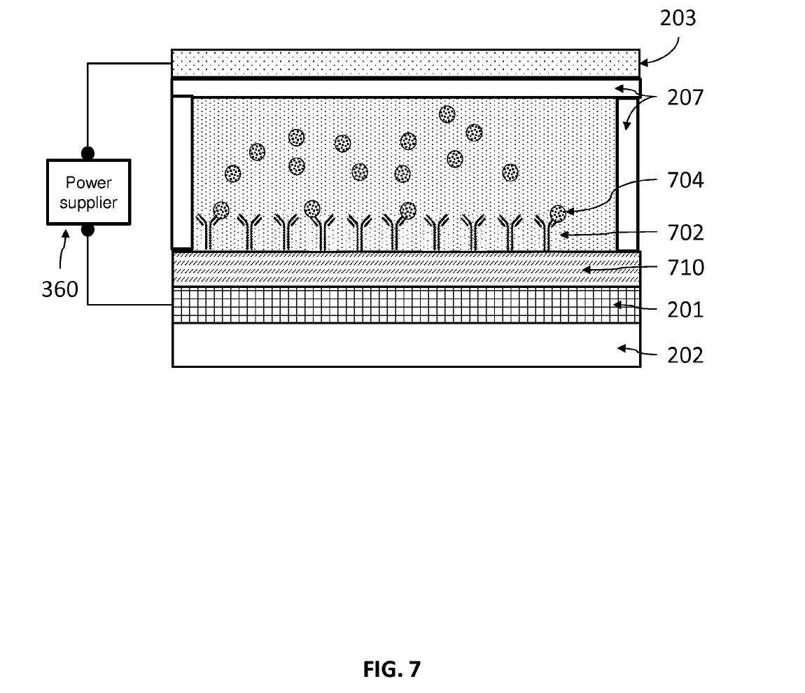

The capture agents for the target analytes are immobilized either directly on the electric field and electric field gradient enhancement Layer (EFGE) or through a thin molecular adhesion/spacer layer (MSL), 710 (FIGS. 7,8, 9).

In one embodiment, the capture agents are attached primarily in the regions of high electric field and/or high electric field, namely, the regions with sharp edges of metallic materials and the small gap. One method of achieving this is to (a) use end functional group in either the capture agent (for direct attachment) or the MSL, that attaches only the metal, and (b) selectively mask the metal surfaces which have a low local electric field or low local electric field gradient (as described in U.S. provisional application Ser. No. 61/801,424, filed on Mar. 15, 2013, and copending PCT application entitled “Methods for enhancing assay sensing properties by selectively masking local surfaces”, filed on Mar. 15, 2014, which are incorporated by reference.

The molecular adhesion/spacer layer (MSL) 710, coated on outer surface of the EFGE (the inner surface of EFGE is the surface in contact with the substrate surface, serves one of the two or both of the functions: (1) provide a good adhesion to bond to the capture agents, and (2) a spacer that control the distance between the metal in the EFGE and the signal generation molecule to optimize signal amplification. One preferred EFGE embodiment is that the dimension of one, several or all critical metallic and dielectric components of EFGE are less than the wavelength of the light in sensing.

Examples for the molecular spacer thickness: The thickness of the spacer (i.e. MSL), that separate the metal from the molecules that generate optical signal, is from 3 nm to 50 nm for fluorescence (preferred for 5 nm for ˜800 nm light wavelength); and 1 to 15 nm for surface enhanced Raman scattering (SERS). The thickness depends on the wavelength of light.

### Examples for EFGE Structures-1. Molecular Adhesion Layer and Attachment of Capture Agents.

In one embodiment, there is a molecular adhesion layer (also termed “molecular linking layer”) (MAL) 710 between the EFGE and the capture agents (See FIGS. 7, 8, 9). The molecular adhesion layer serves two purposes. First, the molecular adhesion layer acts a spacer. For optimal fluorescence, the light-emitting labels (e.g., fluorophores) cannot be too close to the metal surface because non-radiation processes may quench fluorescence. Nor can the light-emitting labels be too far from the metal surface because it may reduce amplification. Ideally, the light-emitting labels should be at an optimum distance from the metal surface. Second, the molecular adhesion layer provides a good adhesion to attach capture agent onto the EFGE layer. Adhesion is achieved by having reactive groups in the molecules of the molecular adhesion layer, which have a high affinity to the capture agent on one side and to the EFGE layer on the other side.

The molecular adhesion layer can have many different configurations, including (a) a self-assembled monolayer (SAM) of cross-link molecules, (b) a multi-molecular layers thin film, (c) a combination of (a) and (b), and (d) a capture agent itself.

Various method for linking capture agents to a metal surface, with or without a molecular linking layer, are described in WO2013154770, which is incorporated by reference for such methods. For example, in some cases, the metal surface may be first joined to one end (e.g., via a thiol or silane head group) of a molecule of a defined length (e.g., of 0.5 nm to 50 nm in length) and the capture agent can be linked to the other end of the molecule via a capture agent-reactive group (e.g., an N-hydroxysuccinimidyl ester, maleimide, or iodoacetyl group). Dithiobis(succinimidyl undecanoate), which has a —SH head group that binds to a gold surface through sulfer-gold bond, and an NHS-ester terminal group that reacts with primary amines, may be used in certain cases.

Electric Bias (i.e. Voltage)

A voltage bias (i.e. voltage difference) can be applied using a power supply, 360. The voltage bias creates electric field and electric field gradient. The voltage can be either DC or AC or combined and alternating. In DC voltage, the amplitude of the voltage is between 0.1 V to 1,000 V, depending upon the gap, depending upon the spacing between the electrodes. A preferred average electric field between the two electrode should be larger than 100V/cm.

In AC bias, the amplitude is 0.1 V to 1000 V (depending upon the spacing between the two electrode) and the frequency is from 100 Hz to 20 MHz. The exact voltage bias to be used depends on the required electric field and/or the electric field gradient.

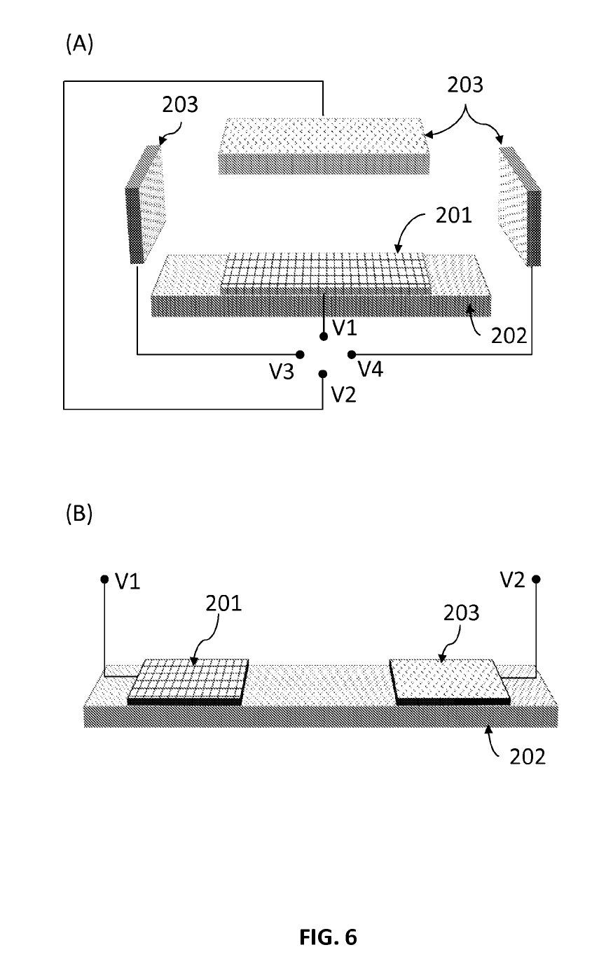

The voltage bias can be applied by using different arrangement of the counter electrodes. Shown in FIG. 1, the counter electrode is on top of the EFGE layer (which is another electrode). In FIG. 6A, the counter electrodes are placed vertically towards the EFGE layer, as well as in the horizontal direction across the substrate. And in FIG. 6B, the counter electrode is in-plane with the EFGE layer.

### Use of Light to Enhance Electric Field and Electric Field Gradient

Another embodiment of the invention is to use light, rather the electric bias, to create the electric field and gradient on the EFGE layer surface. The light absorbed by EFGE will be focused to the sharp edges of the metallic materials and the gaps between two metallic materials, hence creating electric field and gradient which will act on the molecules. The light wavelength will be determined by the resonant wavelength of the EFGE layer and can be from 300 nm to 5000 nm. The preferred wavelength is 400 nm to 1500 nm—visble and near infrared light.

Another embodiment of the invention is that both electrical bias and the light are used together or alternatively to create electric field and gradient on the surface of the EFGE surface.

Another embodiment of the invention is that the same EFGE layer also can directly amplify the sensing signal as well.

### Control of pH of Solution

Another embodiment of the invention is that the control of the pH value of the solution according to isoelectric points of the analytes are used together the electrical bias and/or the light. One key embodiment of the invention is the design and control of the electric field direction and the sample matrix's pH value according to a molecule's isoelectric point (pI) and charge distribution for the transportation, manipulation and orientation of capture agent 206 and target analyte 204, which ensures an experiment that simultaneously satisfy two conditions: (1) the pH value of running buffer solution causes the molecules to carry opposite sign of electric charges to the EFEG layer 201, and (2) the electric field supplied between the EFEG layer 201 and counter electrodes 203 is parallel and aligned to the direction of the biomolecule's electric dipole moment.

### Binding the Analytes

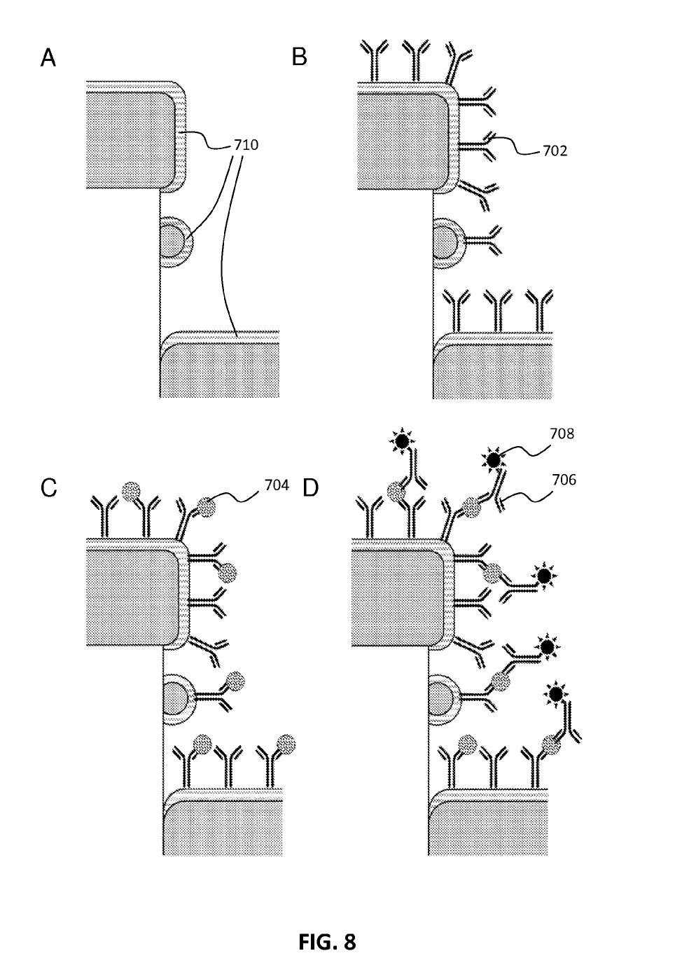

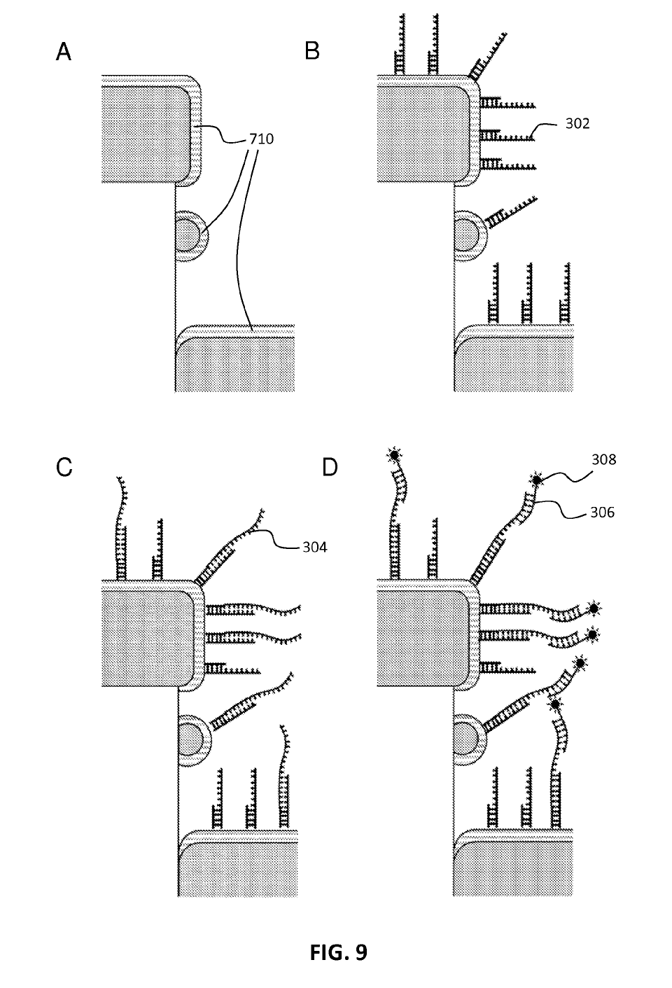

FIG. 8 illustrates a biosensor in which the capture agent is a protein, e.g., an antibody. FIG. 9 illustrates a biosensor in which the capture agent is a nucleic acid, e.g., an oligonucleotide. In some embodiments, the thickness of the molecular adhesion layer is selected to optimize the amplification of the light signal. Depending on how the analyte is labeled, light signal that is amplified may be luminescence (e.g., chemiluminescent or electroluminescent, or fluorescence).

Some of the steps of an exemplary antibody binding assay are shown in FIG. 8. In this assay, the biosensor is linked to an antibody in accordance with the methods described above to produce a biosensor comprises antibodies 702 that are linked to the molecular adhesion layer 710 of the biosensor. After the biosensor has been produced, the biosensor is contacted with a sample containing a target analyte 704 (e.g., a target protein) under conditions suitable for specific binding. The antibodies specifically bind to target analyte in the sample. After unbound analytes have been washed from the biosensor, the biosensor is contacted with a secondary antibody 206 that is labeled with a light-emitting label 708 under conditions suitable for specific binding. After unbound secondary antibodies have been removed from the biosensor, the biosensor may be read to identify and/or quantify the amount of analyte 204 in the initial sample.

Some of the steps of an exemplary nucleic acid binding assay are shown in FIG. 9. In this assay, biosensor is linked to a nucleic acid, e.g., an oligonucleotide in accordance with the methods described above to produce a biosensor that comprises nucleic acid molecules 302 that are linked to the molecular adhesion layer 710. After the biosensor has been produced, the biosensor is contacted with a sample containing target nucleic acid 304 under conditions suitable for specific hybridization of target nucleic acid to the nucleic acid capture agents. The nucleic acid capture agents specifically binds to target nucleic acid 304 in the sample. After unbound nucleic acids have been washed from the biosensor, the biosensor is contacted with a secondary nucleic acid 306 that is labeled with a light-emitting label 308 under conditions for specific hybridization. After unbound secondary nucleic acids have been removed from the biosensor, the biosensor may be read to identify and/or quantify the amount of nucleic acid in the initial sample.

In the embodiments shown in FIGS. 8 and 9, bound analyte can be detected using a secondary capture agent (i.e. the “detection agent”) may be conjugated to a fluorophore or an enzyme that catalyzes the synthesis of a chromogenic compound that can be detected visually or using an imaging system. In one embodiment, horseradish peroxidase (HRP) may be used, which can convert chromogenic substrates (e.g., TMB, DAB, or ABTS) into colored products, or, alternatively, produce a luminescent product when chemiluminescent substrates are used. In particular embodiments, the light signal produced by the label has a wavelength that is in the range of 300 nm to 900 nm). In certain embodiments, the label may be electrochemiluminescent and, as such, a light signal can be produced by supplying a current to the sensor.

In some embodiments, the secondary capture agent (i.e. the detection agent), e.g., the secondary antibody or secondary nucleic acid, may be linked to a fluorophore.

Methods for labeling proteins, e.g., secondary antibodies, and nucleic acids with fluorophores are well known in the art. Chemiluminescent labels include acridinium esters and sulfonamides, luminol and isoluminol; electrochemiluminescent labels include ruthenium (II) chelates, and others are known.

### EXAMPLES

Aspects of the present teachings can be further understood in light of the following examples, which should not be construed as limiting the scope of the present teachings in any way.

### Example-1

### Significant Faster Assay Time on D2PA Using E-Field

As a demonstration, a 1-step direct immunoassay as a model system is used and shown to achieve a short incubation time of 10 minutes when using AC electric field and 3 minutes when using DC electric field.

Preparation of Immunoassay on D2PA Nanodevice.

The D2PA immunoassay consists of two components: (1) the aforementioned D2PA plasmonic nanostructure coated (2) a mixed self-assembled layers of Protein A layer on top of ithiobis succinimidyl undecanoate (DSU). The DSU molecules provide strong cross-link of protein A to gold surface by providing one end of sulfide that strongly binds to gold and the other end of N-hydroxysuccinimide (NHS) ester group that binds well to Protein A's amine group. Fluorescence labeled IgG (pre-labeled) was used as the model antigen in this assay. The fluorescence label is IRDye800CW, whose absorption and emission wavelength are within the localized plasmonic resonance of the plasmonic nanostructure.

For coating DSU SAM and Protein A on the D2PA, freshly fabricated D2PA substrate was first diced into 5 mm×5 mm pieces and immersed in a solution of 0.5 mM DSU (Dojindo, Japan) in 1,4-dioxane (Sigma-Aldrich), and incubated overnight at room temperature in a sealed container. After incubation, the D2PA substrates were rinsed extensively in 1,4-dioxane and dried with argon gas. These DSU coated D2PA substrates were immediately placed in separated wells of a standard 96-well plates (Pierce, USA). They were then immersed in 100 uL of 10 ug/mL Protein A (Rockland Immunochemicals) in phosphate buffered saline (PBS) solution (pH=7.2, Sigma-Aldrich) and incubated in a sealed condition overnight in the fridge at 4 C. The solution was then aspirated and each individual D2PA plate was washed 3 times in washing solution (R&D systems) for 15 minutes each to remove the unbonded protein A. The plates were then gently rinsed in streams of deionized water to remove any salt content. After drying with argon gas, the D2PA immunoassay plate was ready for immediate immunoassay testing or stored at −20 C degree for later use.

IgG labeled with IRdye800CW at a concentration of 10 pM was added to the immunoassay chamber, while the external electric field was switched on. The sample was then incubated for different time from 10s to 1 hour before the electric field was switched off, followed with 3 times of washing in washing solution. A reference sample was also made by simply incubate without electric field for 1 hour at room-temperature—same as conventional immunoassay incubation conditions.

The distance between the electrodes were always controlled at 3 mm. The sample volume was always controlled at 150 uL, while the surface sample area was 5 mm×5 mm.

After washing, fluorescence intensity of the assays that underwent different incubation time was then measured and compared with the reference sample.

### Results.

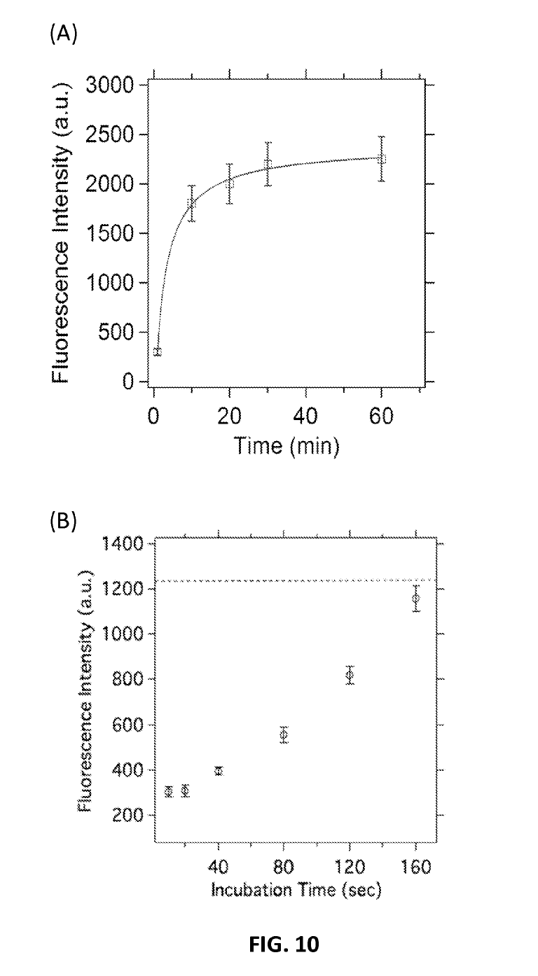

FIG. 10A shows the fluorescence intensity of 10 pM immunoassays that experienced AC field at 250 kHz with Vₚₚ=100V. One can clearly see that at 10 min, the fluorescence intensity start to approaching saturation, which means most of the labeled IgGs has been driven to the plasmonic surface.

FIG. 10B shows the fluorescence intensity of 10 pM immunoassay that experience DC field at V=135 V. One can clearly see that within 160 s (˜3 min) the fluorescence intensity is start to approaching the same value as an 1 hour incubation without any electric field.

### Example-2

### E-Field Reduces D2PA Assay Inter-Assay Variance (CV %) by Enhance Capture Antibody Coating Quality

As a demonstration, a direct 1-step immunoassay is used to demonstrate that using External E-field can significantly improve the capture antibody coating quality by increase the capture efficiency through orientation manipulation.

### Preparation of Immunoassay on D2PA Nanodevice.

The D2PA immunoassay consists of two components: (1) the aforementioned D2PA plasmonic nanostructure coated (2) a mixed self-assembled layers of human IgG layer as the capture antibody on top of ithiobis succinimidyl undecanoate (DSU). The DSU molecules provide strong cross-link of protein A to gold surface by providing one end of sulfide that strongly binds to gold and the other end of N-hydroxysuccinimide (NHS) ester group that binds well to human IgG's amine group. Fluorescence labeled anti-IgG (pre-labeled) was used as the model antigen in this assay. The fluorescence label is IRDye800CW, whose absorption and emission wavelength are within the localized plasmonic resonance of the plasmonic nanostructure.

For coating DSU SAM and IgG on the D2PA, freshly fabricated D2PA substrate was first diced into 5 mm×5 mm pieces and immersed in a solution of 0.5 mM DSU (Dojindo, Japan) in 1,4-dioxane (Sigma-Aldrich), and incubated overnight at room temperature in a sealed container. After incubation, the D2PA substrates were rinsed extensively in 1,4-dioxane and dried with argon gas. These DSU coated D2PA substrates were immediately placed in separated wells of a standard 96-well plates (Pierce, USA). They were then immersed in 100 uL of 1 ug/mL Human IgG (invitrogen) in phosphate buffered (PB) solution (pH=8.0, Sigma-Aldrich) and incubated with changed experimental conditions.

FIG. 9 shows the schematics of experimental principle of this example. Oriented antibody can be immobilized on metallic components:
1. Antibodies carries negative charge in PB buffer (pH=8.0). Therefore the antibodies will be pulled to the electrode when external E-field is applied (FIG. 9A). The amplitude of applied E-field is 27 V/mm. It is preferred that larger E-field is used so that the biomolecules within the buffer solution can feel stronger electrophoresis force. It is advised that E-field larger than breakdown condition between the two electrode be avoided to prevent the nanostructured sensing amplification layer (SAL) on electrode from damage.
2. The heavy chain (Fc regions) of the antibody carries more negative charges than the light chain due to the COO— group. Therefore, antibodies will prefer to bind on the electrode through the heavy chain (FIG. 9B). This stance leads to the vertical orientation of antibodies, which is the preferred orientation for antibodies with maximum capture efficiency.

To compare the effect of E-field on this IgG coating quality, 4 different cases (conditions) were performed:
1. Coating human IgG (concentration: 1 ug/mL) on Protein A (Concentration: 1 ug/mL) coated D2PA without applying any external electric field
2. Directly coating human IgG (concentration: 1 ug/mL) on D2PA with an external electric field with amplitude of 27 V/mm
3. Directly coating human IgG (concentration: 1 ug/mL) on D2PA without applying any external electric field
4. Coating human IgG (concentration: 1 ug/mL) on Protein A (concentration: 1 ug/mL) coated D2PA with an external electric field with amplitude of 27 V/mm

The distance between the electrodes were always controlled at 3 mm. The sample volume was always controlled at 150 uL, while the surface sample area was 5 mm×5 mm. The incubation time was kept at 5 min for all four cases.

After incubation, the solution was then aspirated and each individual D2PA plate was washed 3 times in washing solution (R&D systems) for 15 minutes each to remove the unbonded IgG.

Human anti-IgG labeled with IRdye800CW at a concentration of 200 ng/mL was added to the immunoassay chamber. The sample was then incubated for 30 min before 3 times of washing in washing solution. After rinsing with deionized water and dried with argon gas, the samples with 4 kinds of conditions listed above is measured using fluorescence microscopy to read their signal. To measure the fluorescence intensity, a 785 nm laser with 1.5 mW power is used to excite the fluorescence of immunoassay. A cooled CCD equipped with a spectrometer with spectral resolution of 0.01 nm is used to measure the fluorescence spectrum. The exposure time used for the CCD measurement is 1 second and the fluorescence intensity is calculated as the average count of the fluorescence spectrum within the range of 795 nm to 805 nm, which corresponds to ±5 nm to the emission peak of the NIR fluorescent dye label on the anti-IgG.

Results.

Below is the table summarize the immunoassay results.

| | Case I | Case II | Case III | Case IV |
|---|---|---|---|---|
| E-field (V/mm) | 0 | 27 | 0 | 27 |
| Protein A coating (ug/mL) | 1 | 0 | 0 | 1 |
| Fl. Intensity (a.u.) | 6972 | 7049 | 2667 | 8502 |
| Uniformity (CV %) | 17% | 5% | 26% | 9% |

It is clearly shown that that E-field (E=27 V/mm) assisted antibody coating (human IgG at 1 ug/mL) can achieve equal coating quality to the identical assay that uses Protein A layer (concentration: 1 ug/mL) by comparing the fluorescence intensity of Case I and Case II. It is further shown that the E-field (E=27 V/mm) assisted capture antibody coating (human IgG at 1 ug/mL) can also improve the assay uniformity by 340%, by comparing Case I to Case II (from 17% to 5%). Here the uniformity is the standard deviation of inter-assay results of 5 replicate samples with identical immunoassay and measurement conditions. It is also observed that 20% stronger fluorescence signal can be achieved by using both E-field (E=27 V/mm) and protein A (concentration: 1 ug/mL) coating simultaneously (comparing case IV to case II). However, this may due to the extra spacer of protein A with thickness=4.5 nm, which reduces quenching effect from the metal components of D2PA.

Results.

Below is the table summarize the immunoassay results.

| | Case I | Case II | Case III | Case IV |
|---|---|---|---|---|
| E-field | No | Yes | No | Yes |
| Protein A coating | Yes | No | No | Yes |
| Fl. Intensity (a.u.) | 6972 | 7049 | 2667 | 8502 |
| Uniformity (CV %) | 17% | 5% | 26% | 9% |

It is clearly shown that that E-field assisted antibody coating can achieve equal coating quality to the identical assay that uses Protein A layer by comparing the fluorescence intensity of Case I and Case II. It is further shown that the E-field assisted capture antibody coating can also improve the assay uniformity by 340%, by comparing Case I to Case II (from 17% to 5%). Slightly better fluorescence signal can be achieved by using both E-field and protein A coating, comparing case IV to case II. However, this may due to the extra spacer of protein A with thickness=4.5 nm, which reduces quenching effect from the metal components of D2PA.

### Other Applications

The applications of the subject sensor include, but not limited to, (a) the detection, purification and quantification of chemical compounds or biomolecules that correlates with the stage of certain diseases, e.g., infectious and parasitic disease, injuries, cardiovascular disease, cancer, mental disorders, neuropsychiatric disorders and organic diseases, e.g., pulmonary diseases, renal diseases, (b) the detection, purification and quantification of microorganism, e.g., virus, fungus and bacteria from environment, e.g., water, soil, or biological samples, e.g., tissues, bodily fluids, (c) the detection, quantification of chemical compounds or biological samples that pose hazard to food safety or national security, e.g. toxic waste, anthrax, (d) quantification of vital parameters in medical or physiological monitor, e.g., glucose, blood oxygen level, total blood count, (e) the detection and quantification of specific DNA or RNA from biosamples, e.g., cells, viruses, bodily fluids, (f) the sequencing and comparing of genetic sequences in DNA in the chromosomes and mitochondria for genome analysis or (g) to detect reaction products, e.g., during synthesis or purification of pharmaceuticals.

The detection can be carried out in various sample matrix, such as cells, tissues, bodily fluids, and stool. Bodily fluids of interest include but are not limited to, amniotic fluid, aqueous humour, vitreous humour, blood (e.g., whole blood, fractionated blood, plasma, serum, etc.), breast milk, cerebrospinal fluid (CSF), cerumen (earwax), chyle, chime, endolymph, perilymph, feces, gastric acid, gastric juice, lymph, mucus (including nasal drainage and phlegm), pericardial fluid, peritoneal fluid, pleural fluid, pus, rheum, saliva, sebum (skin oil), semen, sputum, sweat, synovial fluid, tears, vomit, urine and exhaled condensate.

The invention has several key novelties including:
(1) New device platform that can supply uniform electric field across the plasmonic nanostructure for accelerated immunoassay speed. The device platform include a multi-functional electric field supplier, a plasmonic nanostructure device using metals or conducting materials, a chamber on the plasmonic nanostructure for the immunoassay, and a conducting board on top using metals or conducting materials.
(2) New assay structures that give high performances in enhancements of fluorescence and biological/chemical marker detection sensitivity. The new structures include new nanostructures in metals, and dielectric materials or semiconductors, as well as different molecular layers with desired chemical and biological properties.
(3) New plasmonic nanostructures that provide large electric field gradient in the near field, which accelerate the molecules' movement towards the nanogap areas for further fluorescence enhancement.

What is claimed is:

1. A method to reduce assay incubation time and improve the movement, orientation, or bonding of a target analyte in a solution to a sensor, comprising:
(a) obtaining a plate comprising a local electric-field and electric-field gradient enhancement layer on a substrate surface;
(b) attaching capture agents to the surface of the enhancement layer;
(c) applying a voltage between the enhancement layer and at least one counter electrode to produce a local electric field and an electric-field gradient in the solution, wherein the solution is on the plate; and
(d) detecting binding of the target analyte to the capture agents on the plate;
wherein the speed of movement of the analyte, the orientation of the analyte, the orientation of the capture agent, the speed of binding and/or the strength of binding of the analyte to the capture agent are improved by the electric field gradient and/or the electric field, and the time in detecting the analyte is reduced.

2. The method of claim 1, wherein the analyte is selected from the group consisting of a protein, peptide, DNA, RNA, nucleic acid, small molecule, cell, and nanoparticle with different shapes.

3. The method of any prior claim, wherein the method further comprises a step of labeling the target analytes with a label, either prior to or after they are bound to said capture agent.

4. The method of any prior claim, wherein the enhancement layer further enhances the light from the label and/or the light that excites label.

5. The method of any prior claim, wherein the enhancement layer comprises (a) an electrically continuous metallic film and (b) above said metallic film, one or a plural of metallic nanostructures a plurality of which being separated from said metallic film by a distance in the range of 0.5 nm to 100 nm.

6. The method of claim 5, wherein the metallic nanostructures are disks having a shape selected from the group of shapes consisting of round, polygonal, pyramidal, elliptical, elongated bar shaped, or any combination thereof, and the disks have an average lateral dimension in the range of 20 nm to 250 nm.

7. The method of claim 5, wherein the distance is in the range of 0.5 to 30 nm.

8. The method of claim 5, wherein the enhancement layer comprises an electrically continuous metallic film with metallic nanostructures and/or nanoscale voids on the surface and/or inside said metallic film.

9. The method of claim 1, wherein the enhancement layer comprises a D2PA array, wherein the electric field and electrical field gradient are enhanced in the regions of nanostructures and nanogaps of the D2PA array.

10. The method of any prior claim, wherein the enhancement layer directly enhances a signal from the target analytes

11. The method of any prior claim, wherein in the applying a voltage step (c), further comprise a step of shining light on the enhancement layer, wherein the light wavelength is resonant with the enhancement layer to enhance the electric field and the electric field gradient in the region of the nanostructures and the nanogaps.

12. The method of any prior claim, wherein the voltage is set to zero while shining light on the enhancement layer, wherein the light wavelength is resonant with the enhancement layer to enhance the electric field and the electric field gradient in the region of the nanostructures and the nanogaps.

13. The method of any prior claim, wherein in the binding targeted analytes step (d), further comprises a step of controlling of the pH value of solution for reducing incubation time and improving bonding quality.

14. The method of any prior claim, wherein in the attaching the capture agents step (b), further comprising either applying a voltage between the enhancement layer and another electrode, or shining light on the enhancement layer, or both, to reduce incubation time and improve molecular bonding quality.

15. The method of any prior claim, wherein the capture agent specifically binds to an analyte.

16. The method of any prior claim, wherein before the step (b), the method further comprises a step of labeling the target analytes with a label, either prior to or after they are bound to said capture agent.

17. The method of claim 15, wherein during the labeling the target analytes with a label after they are bound to said capture agent, the method further comprises a step of either applying a voltage between the enhancement layer and another electrode, or shining light on the enhancement layer, or both, to reduce labeling incubation time and improve molecular bonding quality.

18. The method of any prior claim, wherein the signals from the target analytes are luminescence that includes fluorescence, electroluminescence, chemiluminescence, and electrochemiluminescence, or Raman scattering.

19. The method of any prior claim, wherein the plate is in a microfluidic channel.

20. The method of any prior claim, wherein the field is a DC field generated by a voltage difference in the range of 1V to 1000V or an AC field generated by a peak to peak voltage difference of 1V to 1000V with a frequency of 1000 kHz to 2 MHz.

21. The method of any prior claim, wherein method comprises binding the analytes to the capture agent, and detecting the analytes using a labeled detection agent.

22. The method of any prior claim, wherein the enhancement layer has a molecular linking layer that links said capture agents with the enhancement layer.

23. A system comprising:
(a) a plate comprises (i) an enhancement layer comprises nanostructures that enhance local electric-fields and electric-field gradients in regions on or near the surfaces of the enhancement layer and (ii) capture agents are attached to said amplification layer;
(b) at least one counter electrode; and
(b) a power supply that connected to the enhancement layer and the at least one counter electrode.

24. The system of claim 23, wherein the enhancement layer comprises (a) an electrically continuous metallic film and (b) above said metallic film, one or a plural of metallic nanostructures a plurality of which being separated from said metallic film by a distance in the range of 0.5 nm to 100 nm.

25. The system of claim 23, wherein the metallic nanostructures are disks having a shape selected from the group of shapes consisting of round, polygonal, pyramidal, elliptical, elongated bar shaped, or any combination thereof, and the disks have an average lateral dimension in the range of 20 nm to 250 nm.

26. The system of claim 23, wherein the distance is in the range of 0.5 to 30 nm.

27. The system of claim 23, wherein the enhancement layer comprises an electrically continuous metallic film with metallic nanostructures and/or nanoscale voids on the surface and/or inside said metallic film.

28. The system of claim 23, wherein the enhancement layer comprises a D2PA array, wherein the electric field and electrical field gradient are enhanced in the regions of nanostructures and nanogaps of the D2PA array.

29. A system comprising:
(a) a plate comprises (i) an enhancement layer comprises nanostructures that enhance local electric-fields and electric-field gradients in regions on or near the surfaces of the enhancement layer and (ii) capture agents are attached to said amplification layer;
(b) a light source that illuminate the enhancement layer.

30. The system of claim 29, wherein the enhancement layer comprises (a) an electrically continuous metallic film and (b) above said metallic film, one or a plural of metallic nanostructures a plurality of which being separated from said metallic film by a distance in the range of 0.5 nm to 100 nm.

31. The system of claim 29, wherein the metallic nanostructures are disks having a shape selected from the group of shapes consisting of round, polygonal, pyramidal, elliptical, elongated bar shaped, or any combination thereof, and the disks have an average lateral dimension in the range of 20 nm to 250 nm.

32. The system of claim 29, wherein the distance is in the range of 0.5 to 30 nm.

33. The system of claim 29, wherein the enhancement layer comprises an electrically continuous metallic film with metallic nanostructures and/or nanoscale voids on the surface and/or inside said metallic film.

34. The system of claim 29, wherein the enhancement layer comprises a D2PA array, wherein the electric field and electrical field gradient are enhanced in the regions of nanostructures and nanogaps of the D2PA array.
